(19) United States
(12) Patent Application Publication
(10) Pub. No.: US 2006/0064134 A1
(43) Pub. Date: Mar. 23, 2006

(54) Systems and methods for deriving relative physiologic measurements

(75) Inventors: Scott T. Mazar, Woodbury, MN (US); Jeffrey A. Von Arx, Minneapolis, MN (US); Abhi Chavan, Maple Grove, MN (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 10/943,626

(22) Filed: Sep. 17, 2004

(51) Int. Cl. A61N 1/36; A61M 31/00

(52) U.S. Cl. 607/17; 607/23; 604/67; 128/903

(57) ABSTRACT

One embodiment of the present invention relates to a system for deriving physiologic measurement values that are relative to ambient conditions. In one embodiment, the system comprises an implantable medical device (“IMD”) and an external monitor. The IMD is adapted to determine an absolute physiologic parameter value within a patient's body, and communicate the absolute physiologic parameter value outside the patient's body, for example, to the external monitor. Further, the external monitor is adapted to receive the absolute physiologic parameter from the IMD and obtain an ambient condition value outside the body that can affect the absolute physiologic parameter value. The external monitor then calculates a relative physiologic parameter value from the ambient condition value and the absolute physiologic parameter value.

### CROSS-REFERENCE TO RELATED APPLICATIONS

The present application is related to three co-pending patent applications: U.S. patent application Ser. No. ______ entitled “SYSTEMS AND METHODS FOR DERIVING RELATIVE PHYSIOLOGIC PARAMETERS USING AN EXTERNAL COMPUTING DEVICE,” and filed by ______ (Attorney Docket No. 306158); U.S. patent application Ser. No. _____ entitled “SYSTEMS AND METHODS FOR DERIVING RELATIVE PHYSIOLOGIC PARAMETERS USING A BACKEND COMPUTING SYSTEM,” and filed by ______ et al. (Attorney Docket No. 306664); and U.S. patent application Ser. No. ______ entitled “SYSTEMS AND METHODS FOR DERIVING RELATIVE PHYSIOLOGIC PARAMETERS USING AN IMPLANTED SENSOR DEVICE,” and filed by ______ et al. (Attorney Docket No. 306663). Each of the above-identified applications is filed on the same date as the present application, and each of the above-identified applications is incorporated by reference herein for all purposes.

### BACKGROUND OF THE INVENTION

The present invention relates generally to measuring physiologic parameters of a patient, and more particularly, to systems, methods and devices for measuring and deriving physiologic parameters relative to various environmental parameters.

Medical devices are known that can be implanted within a patient's body for monitoring one or more physiological parameters and/or to provide therapeutic functions. For example, sensors or transducers can be placed in the body for monitoring a variety of properties, such as temperature, blood pressure, strain, fluid flow, chemical properties, electrical properties, magnetic properties, and the like. In addition, medical devices can be implanted that perform one or more therapeutic functions, such as drug delivery, cardiac pacing, defibrillation, electrical stimulation, and the like.

In many cases, the implanted medical devices (“IMDs”) are configured or adapted to communicate with external controllers or programmers, for example, to communicate data between the IMD and the external programmers, and/or to activate or otherwise control the IMDs. Typically, the IMDs can communicate with the external programmers via a wireless communication link, such as an RF communication link, or the like.

As mentioned above, IMDs can be configured to measure or sense a number of different parameters in the body. One parameter of particular interest is blood pressure. The implantable biosensors that measure pressure deep within anatomical structures, however, typically can only communicate the absolute pressure associated with the immediate anatomical environment. These devices are not capable of communicating gauge pressure because they are confined and sealed away from the ambient pressure external the body. In most cases, it is gauge pressure and not absolute pressure that is sought to be known, since the body regulates its activities based on the ambient pressure. Gauge pressure may be determined by correlating the absolute pressure with the ambient pressure. Similar situations exist for other parameters, as well, such as temperature.

Thus, a need exists for systems, methods, and/or devices for adjusting measured physiologic parameters, such as pressure, temperature and others, based on ambient or other environmental conditions.

### BRIEF SUMMARY OF THE INVENTION

One embodiment of the present invention relates to a system for deriving physiologic measurement values that are relative to ambient conditions. In one embodiment, the system comprises an implantable medical device (“IMD”) and an external monitor. The IMD is adapted to determine an absolute physiologic parameter value within a patient's body, and communicate the absolute physiologic parameter value outside the patient's body, for example, to the external monitor. Further, the external monitor is adapted to receive the absolute physiologic parameter from the IMD and obtain an ambient condition value outside the body that can affect the absolute physiologic parameter value. The external monitor then calculates a relative physiologic parameter value from the ambient condition value and the absolute physiologic parameter value.

In one embodiment, the external monitor then communicates the relative physiologic parameter value to the IMD, which stores it in a memory. In other embodiments, the IMD can be adapted to communicate the relative physiologic parameter value to an external computing device, and/or the external monitor can be adapted to store the relative physiologic parameter value in a memory, as well.

In accordance with another embodiment, the absolute physiologic parameter value and/or the ambient condition value can be marked with a time stamp. Thus, because time stamps are used, the external monitor can select an ambient condition value that is obtained or measured at a time reasonably close to the time when the absolute physiologic parameter value is measured to calculate the relative physiologic parameter value.

In accordance with one embodiment, the absolute physiologic parameter value comprises blood pressure (e.g., intravascular or intracardiac blood pressure). Thus, in accordance with this embodiment, the ambient condition value comprises an ambient pressure value, and the relative physiologic parameter value comprises blood pressure relative to the ambient pressure. For example, the relative physiologic parameter value can be a gauge pressure value calculated by subtracting the ambient pressure value from the blood pressure.

In yet another embodiment, the blood pressure can be determined by measuring the blood pressure with a pressure sensor and adjusting the blood pressure with a sensor specific adjustment factor to obtain an absolute blood pressure value.

In one embodiment, the external monitor can be adapted to measure the ambient condition value, or the external monitor can be adapted to receive the ambient condition value from an ambient condition source. Also, the external monitor can be a wireless communication device carried on or near the patient's body. For example, the external monitor can be a device adapted to be worn around a patient's body part, a device that can be connected to a belt, a device that can be worn as a patch, a personal digital assistant (PDA), a device that can be carried in a pocket or a personal carry bag, a customized cell phone or pager, or any other suitable device that can perform the functions of the external monitor.

In some embodiments, the IMD and the external monitor are adapted to communicate via a wireless communication connection, such as, for example, a radio frequency communication connection, an acoustic communication connection, a magnetic field communication connection, an optical communication connection, or any other suitable wireless communication connection.

The IMD can be any suitable implantable medical device. For example, in some embodiments, the IMD can be a physiologic parameter sensor, a pacemaker, a defibrillator, a bi-ventricular pacer, a ventricular assist blood pump, a drug delivery pump, a drug infusion device, a neurostimulating device, an intra-ocular shunt, an intra-cranial shunt, or any other suitable IMD. Also, the IMD can be adapted to provide therapy to a patient. In accordance with this embodiment, the IMD can be configured to use the relative physiologic parameter value to determine the proper therapy to provide to the patient. For example, in some embodiments, the IMD can use the relative physiologic parameter value to determine and provide therapies, such as, cardiac pacing therapy, anti-tachycardia therapy, drug delivery therapy, neurostimulation therapy, blood pump therapy, or any other suitable therapy provided by IMDs.

In still other embodiments, the IMD can include a processor that can be adapted to generate patient therapy information based at least in part on the relative physiologic parameter value. In accordance with this embodiment, the implantable medical device can be configured to communicate the patient therapy information to the external monitor, and the external monitor can include a patient communication interface adapted to provide the patient therapy information to the patient.

In yet other embodiments, the present invention can include methods performed by the IMD and the external monitor, and the present invention can include the IMDs and/or the external monitors individually.

A more complete understanding of the present invention may be derived by referring to the detailed description of preferred embodiments and claims when considered in connection with the figures.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 is a diagram showing one embodiment of a system of the present invention;

FIG. 2 is a block diagram showing one embodiment of an implantable medical device;

FIG. 3 is a block diagram showing one embodiment of a remote sensor system of an implantable medical device;

FIG. 4 is a block diagram of one embodiment of an external monitor; and

FIGS. 5-12 are flow charts illustrating various embodiments of methods of the present invention.

### DETAILED DESCRIPTION OF THE INVENTION

The present invention relates generally to measuring physiological parameters of a patient, and more particularly, to systems, methods and devices for measuring and deriving physiological parameters relative to various environmental parameters. In contrast to prior art systems and methods, the present invention utilizes one or more external monitoring devices to obtain ambient measurement values, and then the one or more external monitors use the ambient values along with the measured physiologic parameters to calculate relative physiologic parameter values.

As discussed herein, the present invention relates to systems and methods for obtaining relative physiologic parameter values. The physiologic parameter values obtained can be any physiologic measurement, such as, blood pressure, temperature, blood or fluid flow, strain, electrical, chemical or magnetic properties within the body, or the like. As one skilled in the art will appreciate, the type of measurement being taken may determine whether the measurement will need to be adjusted for ambient or other environmental conditions that may affect the measurements within the body. For example, it is known that ambient pressure will affect intravascular blood pressure. Further, outside temperature may affect the internal body temperature of a person or other animal, depending, in part, on the severity of the outside temperature. Also, other environmental conditions, such as, altitude, humidity, electric or magnetic fields, or chemical compositions may affect other physiologic measurements of interest. Accordingly, the present invention can be used to adjust any internal physiologic measurements relative to environmental conditions, and thus, is not limited in scope to any particular measurement. Of course, as one skilled in the art will appreciate, adjusting measured internal blood pressure relative to ambient pressure (which also can be derived from or affected by temperature, humidity, altitude, etc.) is of particular interest for many clinical and therapeutic applications, and the present invention is well suited to obtain relative pressure values.

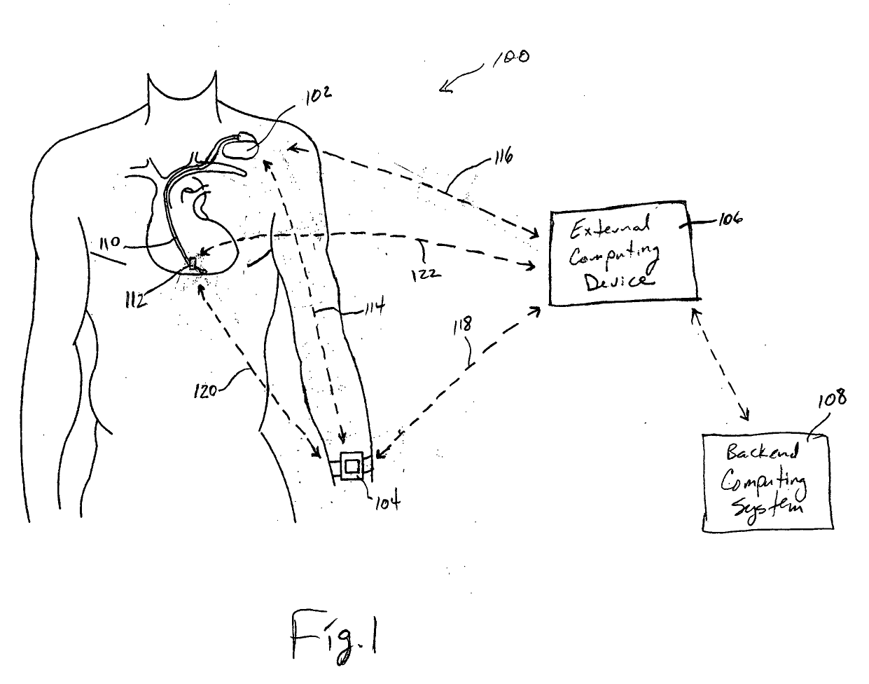

Referring now to FIG. 1, one embodiment of a system 100 for deriving relative physiologic measurement values is shown. In accordance with the illustrated embodiment, system 100 includes an implantable medical device (“IMD”) 102, an external monitoring device 104, an external computing device 106, and a backend computing system 108.

IMD 102 can be any type of implantable medical device that is configured to obtain physiologic measurements of a patient and/or provide therapy to the patient. For example, IMD 102 can be a pacemaker, an implantable cardioverter defibrillator (“ICD”), a cardiac resynchronization device, a bi-ventricular pacer, a ventricular assist blood pump, a drug delivery pump, a drug infusion device, a neurostimulating device, an intra-ocular shunt, an intra-cranial shunt, or any other suitable implantable device, which can obtain or measure physiologic parameter values. In the embodiment illustrated in FIG. 1, IMD 102 is a cardiac device, such as a pacemaker or ICD, which has one or more leads 110 that can include sensors or sensor circuitry 112 for obtaining physiologic measurements (e.g., blood pressure or the like) and therapy delivery systems (also 112) for providing cardiac therapy to the patient.

In addition, in alternative embodiments, sensors and/or therapy delivery systems 112 can be located separate from leads 110, and the sensor data can be transmitted to IMD 102 via other communication medium, such as a coupled communication connection, or a wireless communication connection. If a wireless communication connection is used, the wireless connection can be a radio frequency (RF) connection, an acoustic connection (e.g., ultrasound), an optical connection, an electric field connection, or any other suitable communication connection. For example, in one embodiment, sensors and/or therapy delivery systems 112 can be configured in one or more satellite devices (e.g., chronically implanted stent devices) as is disclosed in Published U.S. Patent Application No. 2003/0158584 A1, published on Aug. 21, 2003, and entitled “Chronically-Implanted Device For Sensing and Therapy,” the entirely of which is incorporated by reference herein for all purposes.

In addition, in some embodiments, IMD 102 is operable to communicate with external monitoring device 104 and external computing device 106 via wireless connections 114 and 116, respectively. Similarly, in some embodiments, external monitoring device 104 is operable to communicate with external computing device 106 via wireless connection 118. Further, in other embodiments, external monitor 104 and external computing device 106 can be in wireless communication with sensor circuitry 112 via wireless connections 120 and 122, respectively. Each of these different embodiments will be discussed in more detail below. Wireless connections 114, 116, 118, 120 and 122 all can be uni-directional or bi-directional communication links, depending on the IMD and the data being transferred. As such, IMD 102 and/or sensor circuitry 112 can send information to and receive information from external monitoring device 104 and external computing device 106 via the communication connections. Similarly, external monitoring device 104 can communicate bi-directionally with external computing device 106.

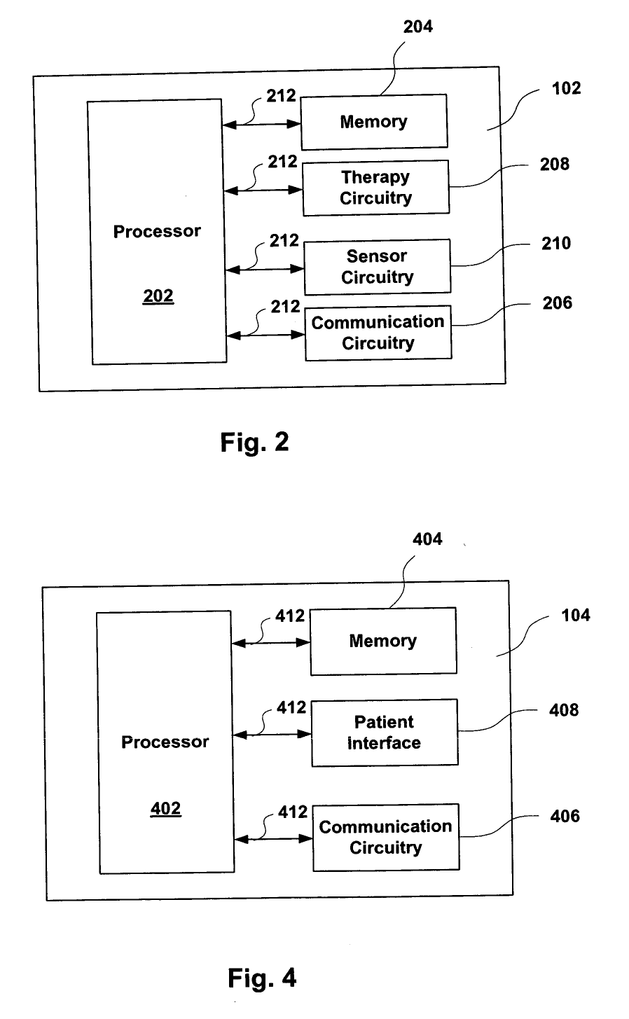

Referring now to FIG. 2, one embodiment of an IMD 102 is illustrated. In accordance with the illustrated embodiment, IMD 102 comprises a processor 202, a memory 204, communication circuitry 206, therapy circuitry 208 and sensor circuitry 210. Memory 204, communication circuitry 206, therapy circuitry 208 and sensor circuitry 210 all are in electrical communication with processor 202, as is illustrated by arrows 212.

As one skilled in the art will appreciate, processors and memory devices are well known in the art, and the specific type and/or style of processor or memory device that can be used in IMD 102 is not limited. Accordingly, processor 202 can be any suitable processing device currently known or hereinafter developed, and memory device 204 can be any suitable memory device currently known or hereinafter developed.

Communication circuitry 206 is circuitry that allows IMD 102 to communicate with other devices, such as external computing device 106, external monitoring device 104, other IMDs, or other external devices. As discussed above, IMD 102 communicates with other devices via a wireless connection. The wireless connection can be, for example, a near field radio frequency (RF) communication connection, a far field RF communication connection, an acoustic communication connection (e.g., an ultrasound connection), an optical communication connection, or any other suitable wireless communication connection.

In one embodiment, communication circuitry 206 can include circuitry for both near field RF telemetry and far field RF telemetry. For example, one embodiment of communication circuitry that can be used in IMD 102 is disclosed in Published U.S. Patent App. No. US 2003/0114897 A1, published on Jun. 19, 2003, and entitled “Implantable Medical Device with Two or More Telemetry Systems,” and Published U.S. Patent App. No. U.S. 2003/0114898 A1, published on Jun. 19, 2003, and entitled “Telemetry Duty Cycle Management System for an Implantable Medical Device,” both of which are incorporated by reference herein for all purposes.

In addition, in other embodiments, power saving wireless communication circuitry and methods can be used. For example, the IMD communication circuitry 206 can be configured to reside in a power-saving, sleep mode for a majority of the time. In accordance with this embodiment, communication circuitry 206 can be configured to “wake-up” on a periodic basis to communicate with an external device. Upon “wake-up” the external device will monitor for RF activity, and if the external device locates it, communication between the IMD and the external device can be initiated. There are a number of different ways IMD power-saving modes can be implemented, and the present invention is not limited to any particular one. Indeed, the aforementioned Published U.S. Patent App. Nos. US 2003/0114897 A1 and US 2003/0114898 A1 disclose different ways of implementing IMD power-saving modes, which, as discussed above, are incorporated herein by reference for all purposes. In addition, additional power management systems and methods are disclosed in Published U.S. Patent App. No. US 2003/0149459 A1, published on Aug. 7, 2003, and entitled “Methods and Apparatuses for Implantable Medical Device Telemetry Power Management,” the entirety of which is incorporated by reference herein for all purposes.

Further, in accordance with other embodiments, communication circuitry 206 can be configured to communicate with an intermediary telemetry device, which, in turn, can facilitate communication with the external monitoring device 104 and/or external computing device 106. One example of this type of configuration is disclosed in Published U.S. Patent App. No. US 2003/0130708, published on Jul. 10, 2003, and entitled “Two-Hop Telemetry Interface for Medical Device,” the entirety of which is incorporated by reference herein for all purposes. In addition, other configurations for RF telemetry are known, and communication circuitry 206 can embody those configurations, as well. Thus, as one skilled in the art will appreciate, communication circuitry 206 is not limited by any particular configuration or communication means.

Therapy circuitry 208 comprises circuitry for providing one or more therapeutic functions to a patient. For example, therapy circuitry 208 can include circuitry for providing heart pacing therapy, cardiac defibrillation therapy, cardiac resynchronization therapy, drug delivery therapy, or any other therapy associated with a suitable IMD. In the case of cardiac therapy (e.g., pacing, defibrillation, etc.), therapy circuitry 208 can include cardiac leads for delivering the therapy to particular locations in the heart. In other embodiments, the therapy circuitry and/or therapy delivery mechanisms can reside in a satellite device wirelessly coupled to the IMD body 102, as discussed below.

Finally, sensor circuitry 210 comprises the sensors and circuitry needed to obtain or measure the physiologic parameters. For example, to obtain a blood pressure (e.g., intravascular or intracardiac blood pressure), sensor circuitry 210 comprises one or more pressure sensors and associated circuitry for recording the pressure accurately. Pressure sensors and the associated circuitry are well known in the art, and therefore, will not be disclosed in detail herein. In addition, in other embodiments, sensor circuitry 210 can be configured to obtain other physiologic parameters, such as temperature, electrical impedance, position, strain, pH, fluid flow, blood oxygen levels, and the like. In these cases, sensor circuitry 210 will include suitable bio-sensors for obtaining the corresponding physiologic parameters.

Also, as one skilled in the art will appreciate, the sensors and/or sensor circuitry can be, and many times are, electrically coupled to IMD 102, but placed remotely from the IMD; e.g., at the end of a lead or in a satellite device in wireless communication with IMD 102. FIG. 1 illustrates an embodiment in which the sensors and/or therapy delivery mechanisms 112 are connected to IMD 102 via leads 110.

In an alternative embodiment, IMD 102 can comprise a planet/satellite configuration, in which the satellite portion of the IMD includes sensor and/or therapy delivery circuits and mechanisms. Such a configuration is disclosed in Published U.S. Patent Application No. US 2003/0158584 A1, published on Aug. 21, 2003, and entitled “Chronically-Implanted Device for Sensing and Therapy,” the entirety of which is incorporated herein by reference for all purposes. In this system, the planet or main body of the IMD communicates with one or more satellite sensor/therapy devices either by an electrical wire connection or wirelessly. In some embodiments, the planet or main body can command each satellite to provide sensing functions and therapy functions, such as delivering cardiac electrical pulses, drug delivery, or other functions, as discussed above. In other embodiments, the satellite devices can function autonomously, and then communicate with the planet device at their own direction, at the planet's direction, or at timed intervals. The relationships between the planet device and the satellite device(s) are discussed in more detail in the incorporated reference.

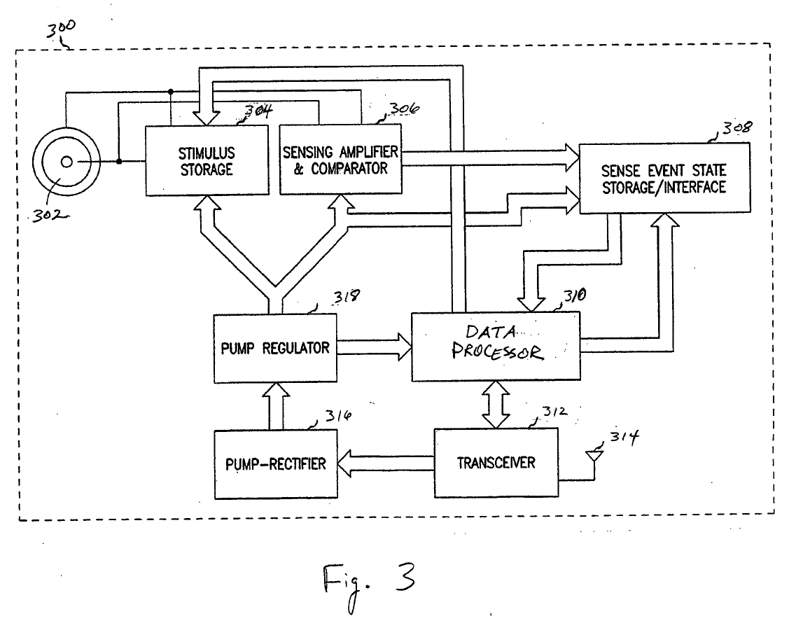

In accordance with one embodiment of the invention, the satellite sensor/therapy devices can be configured as stent-like, intraveous, chronically-implanted devices. The configuration of embodiments of stent-like devices is disclosed in more detail in the incorporated Published U.S. Patent Application No. US 2003/0158584. FIG. 3, however, illustrates one embodiment of sensing and/or therapy circuitry 300 of a satellite device. In accordance with the illustrated embodiment, the circuitry 300 comprises a pair of electrodes 302, a stimulus storage unit 304, a sensing amplifier and comparator logic 306, a sense event state storage and interface 308, a data processor 310, a transceiver 312, an antenna 314, a pump-rectifier 316, and a pump-regulator 318. According to various embodiments, the satellite is designed for mechanical, electrical and/or chemical sensing, and for mechanical, electrical and/or drug-eluting therapies.

The operation of circuitry 300 is described in greater detail in the incorporated Published U.S. Patent Application No. US 2003/0158584. However, in accordance with one embodiment, antenna 314 and transceiver 312 can be configured to communicate with the planet or main body of the IMD 102, with external monitor 104, or with external computing device 106. Such communication connections can be RF connections, acoustic connections (e.g., ultrasound), optical connections, or other suitable communication connections. In addition, antenna 314 and transceiver 312 can be configured to communicate with the multiple devices using one or more different connection types.

As discussed in more detail below, in some embodiments, sensor/therapy circuitry 300 is operable to receive ambient condition values, for example, from the main body of the IMD 102, external monitor 104 or external computing device 106 and calculate a relative physiologic parameter value using the ambient condition value. In accordance with these embodiments of the invention, data processor 310 can be configured to calculate the relative physiologic parameter values using measured values and the received ambient condition values. In addition, data processor 310 is operable to format data received by circuitry 300, as well as format data being transmitted by the satellite device. Again, a more complete description of the operation and configuration of the satellite device and associated circuitry is set forth in the incorporated reference.

Referring again to FIG. 1, external monitoring device 104 comprises a device that is adapted to obtain an ambient condition value or other environmental condition value outside the body that can affect the measured physiologic parameter value, and then, in some embodiments, calculate a relative physiologic parameter value using the measured physiologic parameter value and the ambient condition value. For example, as discussed in more detail below, when determining intravascular blood pressure, external monitoring device 104 can calculate a gauge blood pressure by adjusting the measured blood pressure value with an atmospheric or barometric pressure value.

External monitoring device 104 can comprise any type of processor-based device that can be carried on or near a patient's body. For example, external monitoring device 104 can be a device that is adapted to worn around a part of a patient's body, such as a wrist watch, a device connected to a belt, or some other device adapted to be worn around an ankle, leg, arm, neck, chest, stomach, etc. In addition, in other embodiments, external monitoring device can be a computing device worn as a patch, or a computing device that can be carried in a pocket, on a belt clip, or in a personal carry bag, such as a PDA, or a customized cellular phone or pager. One skilled in the art will appreciate that other device can be used as well.

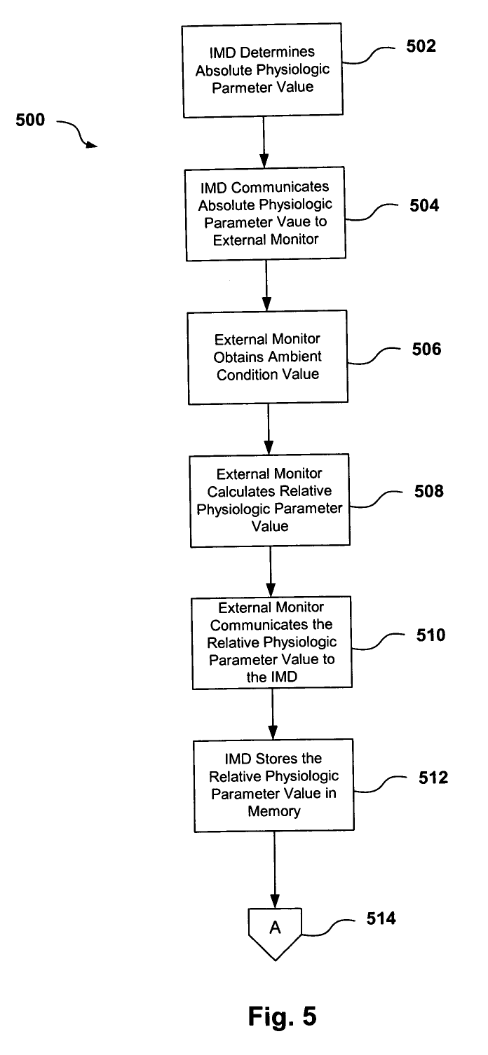

Referring now to FIG. 4, one embodiment of an external monitoring device 104 is shown. In the illustrated embodiment, external monitoring device 104 comprises a processor 402, a memory 404, communication circuitry 406 and a patient interface 408. Memory 404, communication circuitry 406, and patient interface 408 all are in electrical communication with processor 402, as is illustrated by arrows 412.

As one skilled in the art will appreciate, and as discussed above with reference to IMD 102, processors and memory devices are well known in the art, and the specific type and/or style of processor or memory device that can be used in external monitoring device 104 is not limited. Accordingly, processor 402 can be any suitable processing device currently known or hereinafter developed, and memory device 404 can be any suitable memory device currently known or hereinafter developed. In addition, communication circuitry 406 is circuitry that allows external monitoring device 104 to communicate with IMD 102 and other devices, such as external computing device 106, and/or sensor/therapy circuitry 112. Thus, if IMD 102 and the other devices are communicating via an RF connection, communication circuitry 406 comprises RF communication circuitry, as well. Similarly, if optical or acoustic communication connections are used, communication circuitry 406 is adapted to facilitate such connections. Further, in some embodiments, IMD 102 might be communicating with external monitor 104 via one type of communication connection, while other devices, such as external computing device 106 or sensor/therapy circuitry 112 might be communicating with external monitor 104 via other types of connections. Thus, communication circuitry 406 can be any circuitry adapted to facilitate the communications with IMD 102, external computing device 106, and sensor/therapy circuitry 112, and communication circuitry 406 can facilitate multiple types of communications at the same time. As one skilled in the art will appreciate, such circuitry is known in the art, and therefore, will not be discussed in detail herein.

In one embodiment, external monitoring device 104 also can include a patient interface 408 adapted to communicate information, such as therapy or diagnostic information, to the patient. In accordance with this embodiment, patient interface 408 may comprise a visually readable screen that allows the patient to read information, an audible or sound interface that can broadcast or transmit audible or sound information to the patient, or some other communication means.

Finally, referring again to FIG. 1, external computing device 106 can be any suitable computing device adapted to communicate with IMD 102, external monitoring device 104, and/or sensor/therapy circuitry 112, and process data from those devices. For example, in the case of a cardiac rhythm management (“CRM”) IMD (e.g., pacemaker, ICD, etc.), external computing device 106 might be a programmer used by physicians, specialists, or other health care providers extract data from the cardiac IMDs. Programmers are well known in the art. In addition, in other embodiments, external computing device 106 can be a repeater device associate with a patient. Examples of one or more repeater-type devices are disclosed in U.S. Pat. No. 6,607,485, issued on Aug. 9, 2003, and entitled “Computer Readable Storage Medium Containing Code for Automated Collection and Analysis of Patient Information Retrieved from an Implantable Medical Device for Remote Patient Care,” the entirety of which is incorporated by reference herein for all purposes. In still other embodiments, external computing device 106 can be any other computing device for receiving, processing and/or storing medical data, including a workstation or appliance on a network, a server on a network, or a computer network as a whole. The present invention is not limited to any particular type of computing device.

Backend computing system 108 can be any type of computing system. In one embodiment, backend computing system is a networked system with a database operable to receive information regarding patients and their IMDs. The information then can be accessed by third parties, such as physicians, specialists, IMD manufacturers, insurance companies, pharmaceutical companies, or any other health care provider. As discussed in more detail below, backend computing system 108 can be configured to receive relative physiologic parameter values for patients and IMDs, other IMD information, and/or patient therapy information. In addition, backend computing system 108 can be configured to receive physiologic measurements and calculate relative measurements using ambient condition values. Further, backend computing system can determine patient therapy information based on data received from IMD 102, external monitor 104, and/or external computing device 106. Examples of embodiments of a one or more backend computing systems that can be used with the present invention are disclosed in U.S. Pat. No. 6,607,485 discussed above, and U.S. patent application Ser. No. 10/789,964, filed on Feb. 27, 2004, and entitled “Systems and Methods for Automatically Collecting, Formatting and Storing Medical Device Data in a Database,” both of which are incorporated by reference herein for all purposes.

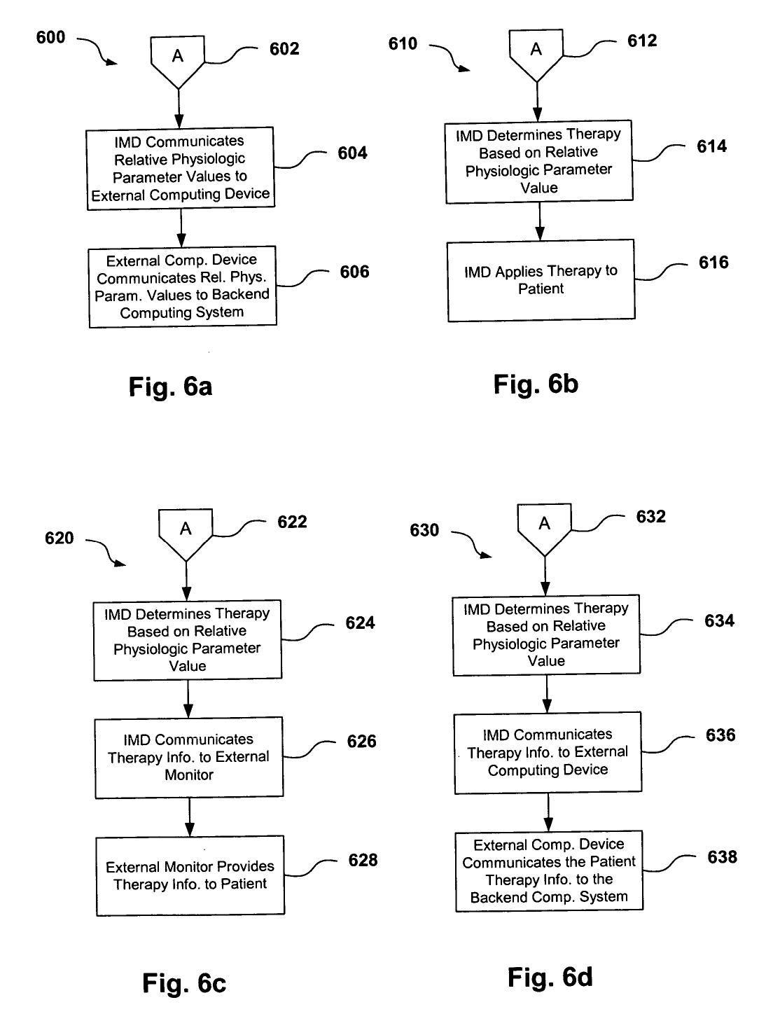

Referring now to FIG. 5, flow chart 500 illustrates a method for obtaining and using relative physiologic parameter values in accordance with one embodiment of the present invention. In accordance with the method illustrated in flow chart 500, an IMD (e.g., IMD 102 in FIG. 1) determines an absolute physiologic parameter value (block 502). As discussed above, the physiologic parameter values obtained can be any physiologic measurement, such as, blood pressure, temperature, blood or fluid flow, strain, electrical, chemical or magnetic properties within the body, or the like. In one particular embodiment, the physiologic parameter value obtained is blood pressure (e.g., intravascular or intracardiac blood pressure), and one or more pressure sensors are used to obtain the absolute blood pressure value. As one skilled in the art will appreciate, the absolute pressure measured by a pressure sensor typically is not a true absolute pressure. The measured pressure typically is adjusted for internal temperatures and/or a built-in backing pressure and/or temperature coefficients of the pressure sensor. Thus, an absolute blood pressure value can be calculated according to the following formula:
P_{ABP}=P_{MBP}−P_{Ref}(T_{Blood}) where:
P_{ABP}is the absolute blood pressure;
P_{MBP} is the measured blood pressure; and
P_{Ref}(T_{Blood}) is the sensor reference/backing pressure, which is a function of blood temperature (T_{Blood}).

As one skilled in the art will appreciate, most pressure sensors are configured to perform these adjustments automatically, and thus, special processing within the IMD typically is not needed to obtain the absolute blood pressure value.

After the IMD obtains the absolute physiologic parameter (e.g., absolute blood pressure), the IMD communicates the absolute physiologic parameter value to an external monitor (e.g., external monitor 104 in FIG. 1) (block 504). In one embodiment, the IMD and the external monitor can implement a data transmission check (e.g., a cyclic redundancy code (“CRC”) check) to ensure the data is transmitted properly.

The external monitor receives the absolute physiologic parameter value and obtains an ambient condition value (block 506). In some embodiments, the external monitor can obtain the ambient condition value either prior to or after receiving the absolute physiologic parameter value. In one embodiment, the external monitor includes a sensor for measuring the ambient condition value. Alternatively, in another embodiment, the external monitor could receive the ambient condition value from another source, such as another computing device in communication with the external monitor, the Internet, or some other source. The means by which the external monitor obtains the ambient condition value is not critical, and thus, the present invention is not limited to any particular method or system for obtaining the ambient condition value.

In some embodiments, the step of determining the absolute physiologic parameter value and the step of obtaining the ambient condition value may not occur at the same time or even close to the same time. For example, the external monitor may only measure or obtain the ambient condition value a few times a day, or there may be a delay between when the external monitor receives the ambient condition value and the absolute physiologic parameter value. As one skilled in the art will appreciate, ambient condition values can change relatively quickly, and thus, if an ambient condition value is not obtained at a time relatively close to when the absolute physiologic parameter value is obtained, the calculated relative value may be inaccurate. Therefore, the absolute physiologic parameter value and/or the ambient condition value can include a time stamp, so that values measured or obtained at about the same time can be used to calculate the relative physiologic parameter value. In some embodiments, if there is a gap between when ambient condition values are measured or obtained, averaged or pro rated ambient values can be used.

As discussed above, in one embodiment, the systems and methods of the present invention can be used to obtain blood pressure. In accordance with this embodiment, the ambient condition value is atmospheric or barometric pressure (and/or other parameters, such as temperature, humidity, altitude, etc. that can affect barometric pressure), which can be measured by one or more sensors in the external monitor, or which can be passed to the external monitor from an outside source. As with the pressure sensor associated with the IMD, a pressure sensor in the external monitor also typically is adjusted for temperature and a built-in backing pressure. Thus, in one embodiment, the atmospheric pressure obtained by the external monitor can be calculated according to the following formula:
P_{ATM}=P_{MATM}−P_{ATM Ref}(T_{ATM}) where:
P_{ATM} is the adjusted atmospheric pressure;
P_{MATM} is the measured atmospheric pressure; and
P_{ATM Ref}(T_{ATM}) is the sensor reference/backing pressure, which is a function of the temperature where the external monitor is placed (T_{ATM}).

As discussed above, most pressure sensors are configured to perform these adjustments automatically, and thus, special processing within the external monitor typically is not needed to obtain the adjusted atmospheric pressure.

After the external monitor obtains the ambient condition value and the absolute physiologic parameter value, the external monitor calculates a relative physiologic parameter value (block 508). For example, for intravascular blood pressure, a gauge blood pressure can be calculated by subtracting the atmospheric pressure from the absolute pressure value.

When pressure sensor adjustments are taken into account, the following formula will apply:
P_{Gauge}=(P_{MBP}−P_{Ref}(T_{Blood}))−(P_{MATM}−P_{ATM Ref}(T_{ATM}))

In other embodiments, the relative physiologic parameter values can be calculated using other mathematical operations or models. For example, in one embodiment, some vascular abnormalities, which can be precursors to some cardiac problems, can be diagnosed by measuring physiologic parameters in two or more locations within the body, using two or more separate sensors. The data from the multiple sensors then might undergo mathematical manipulation, which then can be reconciled with the external ambient condition measurements. Thus, the present invention is not limited to a pure subtraction operation. Further, as one skilled in the art will appreciate, the calculations needed to obtain relative physiologic parameter values from other types of physiologic measurements (e.g., temperature, pH, etc.) may be different depending on the measurements being taken. Also, as discussed briefly above, if the physiologic parameter value and/or the ambient condition value include time stamps, the external monitor will values that are similar in time (or averaged or pro rated values), so that the relative value is accurate.

In one embodiment, after the external monitor calculates the relative physiologic parameter value, it communicates the relative value to the IMD (block 510), which, in turn, stores the relative physiologic parameter value in a memory (block 512). Again, the IMD and the external monitor can implement a data transmission check, such as a CRC check, to ensure the data is transmitted properly. After the relative physiologic parameter value is stored in the IMD memory, the IMD and/or the external monitor can perform a number of different operations with the relative value. This is illustrated by continuation block 514 in FIG. 5.

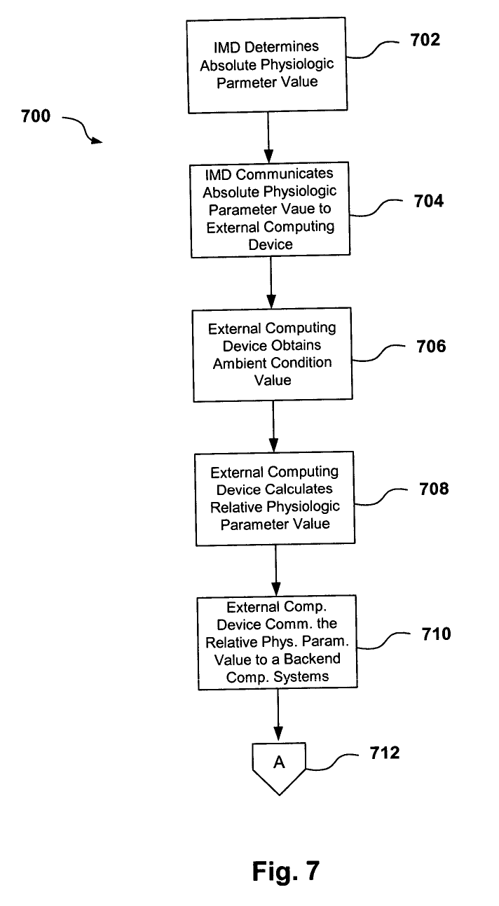

Referring now to FIGS. 6a-6d, additional embodiments of the present invention will be described with reference to flow charts 600, 610, 620 and 630, respectively. In FIGS. 6a-6d, continuation blocks 602, 612, 622 and 632 are continuations from block 514 in FIG. 5. Thus, in accordance with the embodiment illustrated in FIG. 5a, after the IMD stores the relative physiologic parameter value in memory, the IMD can be configured to communicate the relative physiologic parameter value to an external computing device, such as external computing device 106 discussed above with reference to FIG. 1 (block 604). For example, in the case in which the IMD is a CRM device, such as a pacemaker or an ICD, the IMD can communicate the relative physiologic measurements to an external programmer or repeater device along with other IMD data, which is well known. A physician or other health care provider can analyze the relative physiologic measurements to determine trends and provide diagnosis and treatment based on those trends. In addition, the data can be uploaded to one or more backend systems (e.g., backend system 108) for later processing, access, and/or analysis by one or more health care providers, such as physicians, specialists, IMD manufacturers, insurance companies, pharmaceutical companies, or any other suitable health care provider (block 606).

Referring now to FIG. 6b, in this particular embodiment, the IMD can be configured to calculate or determine a therapy based on the relative physiologic parameter value (block 614), and then administer the therapy to the patient (block 616). For example, if the IMD is a cardiac pacemaker, the pacemaker may be configured to change pacing modes or parameters based on the obtained relative physiologic value. Similarly, if the IMD is a drug delivery device, the device might deliver a drug dosage when certain relative physiologic parameter values are measured. As one skilled in the art will appreciate, the type of therapy administered to the patient will be dependent upon the type of IMD used and the physiologic measurements taken. Thus, as discussed above, the present invention is not limited to any particular IMD or therapy system.

Referring now to FIG. 6c, in this particular embodiment, the IMD can be configured to calculate or determine a therapy or therapy information based on the relative physiologic parameter value (block 624). The IMD then is configured to communicate the therapy information to the external monitor (e.g., external monitor 104) (block 626), which in turn can be configured to provide or communicate the therapy information to the patient (block 628). For example, in the case of a CRM IMD, which is configured to detect blood pressure, when the IMD detects a consistent rise in blood pressure for a congestive heart failure patient, the IMD can be configure to communicate information to the patient on how to deal with the situation. For example, the IMD might communicate a message to the external monitor, instructing the patient to change a medication dosage, or perhaps take a medication. As discussed above, the external monitor can include a communication interface, such as a visual screen or audible interface, to communicate the information to the patient.

Referring now to FIG. 6d, in this particular embodiment, the IMD again can be configured to calculate or determine a therapy or therapy information based on the relative physiologic parameter value (block 634). Then, in stead of, or in addition to administering the therapy to the patient, the IMD is configured to communicate the therapy information to an external computing device, such as computing device 106 (block 636). A physician or other health care provider then can analyze the therapy information and provide it to the patient if the physician determines that it is appropriate, or the physician can provide different or additional therapy to the patient if the physician determines that an alternative therapy would be better. In addition, the therapy information can be uploaded to a backend computing system (e.g., backend computing system 108) for later processing, access and/or analysis.

In an alternative embodiment, instead of the external monitor communicating the relative physiologic parameter value to the IMD, it can be configured to communicate the relative physiologic parameter value to the external computing device without first communicating it to the IMD. The external computing device then can communicate it to a backend computing system, or it can display it to the patient or another health care provider. In addition, instead of the IMD determining the therapy information, the external monitor or the external computing device can be configured to determine the therapy information based on, in part, the calculated relative physiologic parameter value. In this embodiment, the processor within the external monitor or the external computing device will include the therapy logic, not the processor within the IMD. In addition, as one skilled in the art will appreciate, the therapy and/or diagnosis information that the IMD, the external monitor, and/or the external computing device provide to the patient will depend upon the type of IMD and the type of physiologic parameters being measured. As discussed above, the present invention is not limited to any particular IMD or physiologic parameter value measurement.

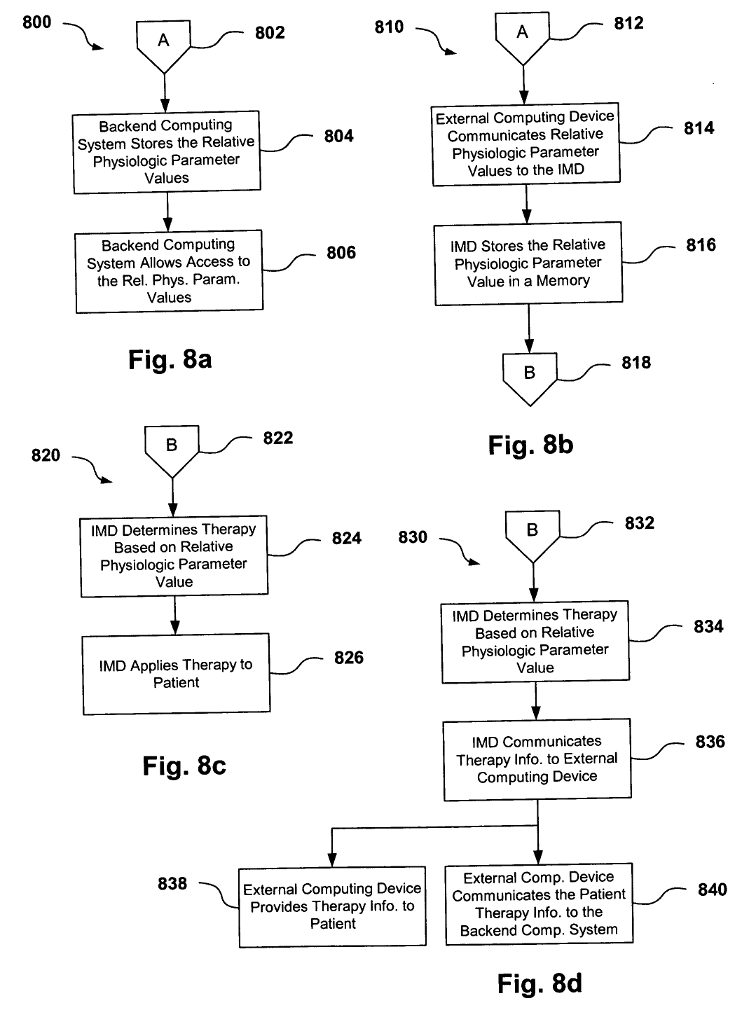

Referring now to FIG. 7, an alternative embodiment of a method of deriving and using relative physiologic parameter values is shown. In accordance with the method illustrated in flow chart 700, an IMD (e.g., IMD 102 in FIG. 1) determines an absolute physiologic parameter value (block 702). As discussed above, the physiologic parameter values obtained can be any physiologic measurement, such as, blood pressure, temperature, blood or fluid flow, strain, electrical, chemical or magnetic properties within the body, or the like. In one particular embodiment, the physiologic parameter value obtained is blood pressure (e.g., intravascular or intracardiac blood pressure), and one or more pressure sensors are used to obtain the absolute blood pressure value. As discussed above, the absolute pressure measured by a pressure sensor can be adjusted for internal temperatures and/or a built-in backing pressure and/or temperature coefficients of the pressure sensor.

After the IMD obtains the absolute physiologic parameter (e.g., absolute blood pressure), the IMD communicates the absolute physiologic parameter value to an external computing device (e.g., external computing device 106 in FIG. 1) (block 704). In one embodiment, the IMD and the external computing device can implement a data transmission check (e.g., a cyclic redundancy code (“CRC”) check) to ensure the data is transmitted properly.

The external computing device receives the absolute physiologic parameter value and obtains an ambient condition value (block 706). In one embodiment, the external computing device includes one or more sensors for measuring the ambient condition value. Alternatively, in another embodiment, the external computing device can receive the ambient condition value from another source, such as another computing device in communication with the external computing device, the Internet, or some other source. For example, as discussed above, an external monitor (e.g., external monitor 104 in FIG. 1) can include a sensor for measuring the ambient condition value, and the external monitor can pass the ambient condition value to the external computing device via a wired or wireless communication connection. The means by which the external computing device obtains the ambient condition value is not critical, and thus, the present invention is not limited to any particular method or system for obtaining the ambient condition value.

In one embodiment, the systems and methods of the present invention can be used to obtain blood pressure. In accordance with this embodiment, the ambient condition value is atmospheric or barometric pressure, which can be measured by one or more sensors in the external computing device, or which can be passed to the external computing device from an outside source (e.g., from an external monitor). As with the pressure sensor associated with the IMD, a pressure sensor in the external computing device or the external monitor also typically is adjusted for temperature and a built-in backing pressure. As mentioned above, most pressure sensors are configured to perform these adjustments automatically, and thus, special processing within the external computing device or the external monitor typically is not needed to obtain the adjusted atmospheric pressure.

After the external computing device obtains the ambient condition value, the external computing device calculates a relative physiologic parameter value (block 708). In one embodiment, for blood pressure, a gauge blood pressure can be calculated by subtracting the atmospheric pressure from the absolute pressure value, or as discussed above, other mathematical calculations can be used. In addition, the calculations needed to obtain relative physiologic parameter values from other types of physiologic measurements (e.g., temperature, pH, etc.) may be different depending on the measurements being taken. Further, as discussed above, the physiologic parameter value and/or the ambient condition value can include time stamps, so that the relative physiologic parameter value is calculated using values that are similar in time.

In one embodiment, after the external computing device calculates the relative physiologic parameter value, it communicates the relative value to a backend computing system (e.g., backend computing system 108 in FIG. 1) (block 710). Further, the external computing device can display or provide the relative physiologic measurement values to a health care provider or other entity for review. For example, in the case in which the IMD is a CRM device, such as a pacemaker or an ICD, the external computing device might be an external programmer with print-out or display capabilities. Thus, a physician or other health care provider can view and analyze the relative physiologic measurements to determine trends and provide diagnosis and treatment based on those trends.

After the relative physiologic parameter value is communicated to the backend computing system, additional processing can occur in a number of different devices in the system. This is illustrated by continuation block 712 in FIG. 7.

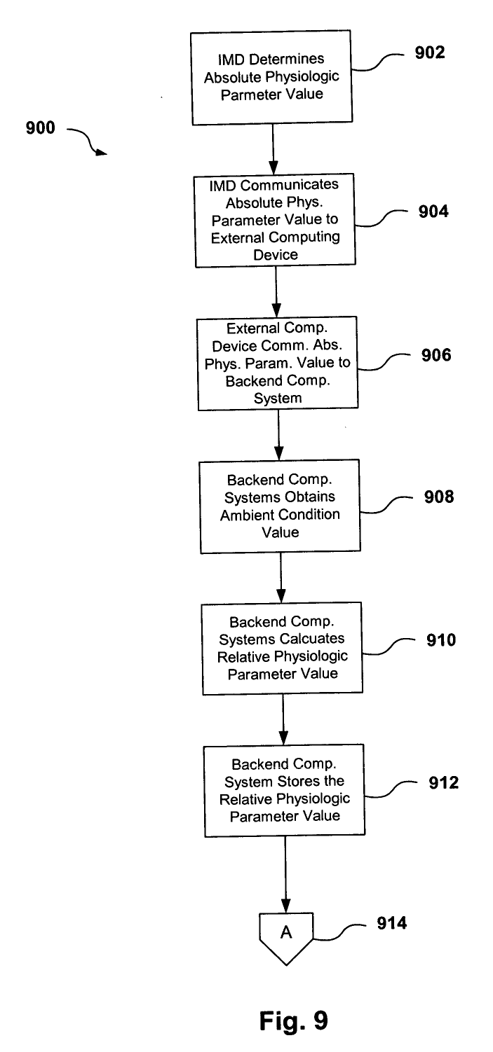

Referring now to FIGS. 8a-8d, additional embodiments of the present invention will be described with reference to flow charts 800, 810, 820 and 830, respectively. In FIGS. 8a-8b, continuation blocks 802 and 812 are continuations from block 712 in FIG. 7. Thus, in accordance with the embodiment illustrated in FIG. 8a, after the external computing device communicates the relative physiologic parameter value to the backend computing system, the backend computing system stores the relative physiologic parameter values, for example, in a database (block 804). The data then can be made available for later processing, access, and/or analysis by one or more health care providers, such as physicians, specialists, IMD manufacturers, insurance companies, pharmaceutical companies, or any other suitable health care provider (block 806).

As illustrated by flow chart 810 in FIG. 8b, the external computing device also can communicate the relative physiologic parameter value to the IMD (block 814), which then stores the relative physiologic parameter value in memory (block 816). When the IMD receives the relative physiologic parameter value, the IMD then can use the value for additional processing, as illustrated by continuation block 818. Continuation blocks 822 and 832 in FIGS. 8c and 8d, respectively, continue from block 818. Also, even though the drawings illustrate that the relative physiologic parameter value is communicated to the backend computing system and then to the IMD, the present invention is not limited to this embodiment. In some embodiments, the external computing device can display the relative physiologic parameter value to a physician or other user, or it can determine therapy information using the relative physiologic parameter value without first communicating the relative value to the backend computing system. Further, the external computing device can communicate the relative physiologic parameter value to the IMD without first communicating it to the backend computing system. Thus, as one skilled in the art will appreciate, the present invention is not limited to the illustrated embodiments.

Referring now to FIG. 8c, in this particular embodiment, the IMD can be configured to calculate or determine a therapy based on the relative physiologic parameter value (block 824). In one embodiment, the IMD can be configured to administer the therapy to the patient (block 826), as discussed above.

Referring now to FIG. 8d, in this particular embodiment, the IMD again can be configured to calculate or determine a therapy or therapy information based on the relative physiologic parameter value (block 834). Then, in stead of, or in addition to administering the therapy to the patient, the IMD is configured to communicate the therapy information to the external computing device (e.g., external computing device 106), or alternatively, the external monitor (e.g., external monitor 104) (block 836). In one embodiment, the external computing device (and/or external monitor) can be configured to provide or communicate the therapy information to the patient via a user or patient interface, as discussed above (block 838).

In an alternative embodiment, after the external computing device (or external monitor) receives the therapy information from the IMD, a physician or other health care provider then can analyze the therapy information and provide it to the patient if the physician determines that it is appropriate, or the physician could provide different or additional therapy to the patient if the physician determines that an alternative therapy would be better. In addition, the therapy information can be uploaded to a backend computing system (e.g., backend computing system 108) (block 840) for later processing, access and/or analysis. Also, instead of the IMD determining the therapy information, the external computing device, the external monitor, and/or the backend computing system can be configured to determine the therapy information based on the relative physiologic parameter value. The present invention is not limited to the IMD determining the therapy.

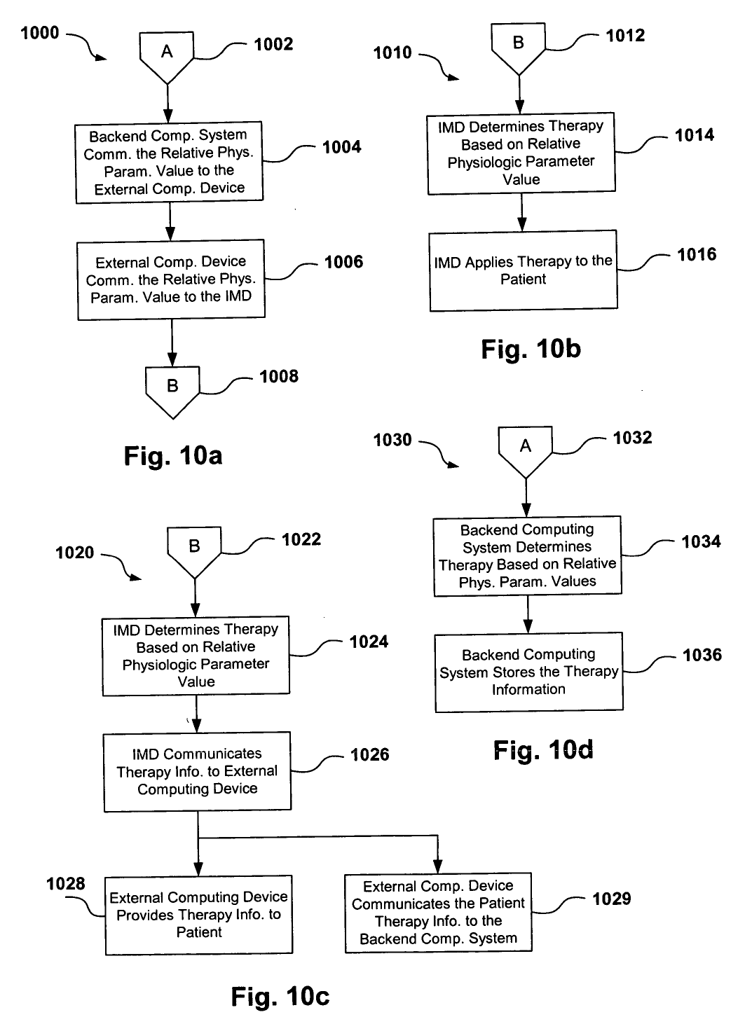

Referring now to FIG. 9, yet another embodiment of a method of deriving and using relative physiologic parameter values is shown. In accordance with the method illustrated in flow chart 900, an IMD (e.g., IMD 102 in FIG. 1) determines an absolute physiologic parameter value (block 902). As discussed above, the physiologic parameter values obtained can be any physiologic measurement, such as, blood pressure, temperature, blood or fluid flow, strain, electrical, chemical or magnetic properties within the body, or the like.

After the IMD obtains the absolute physiologic parameter (e.g., absolute blood pressure), the IMD communicates the absolute physiologic parameter value to an external computing device (e.g., external computing device 106 in FIG. 1) (block 904). Again, the IMD and the external computing device can implement a data transmission check (e.g., a cyclic redundancy code (“CRC”) check) to ensure the data is transmitted properly.

The external computing device receives the absolute physiologic parameter value and communicates it to a backend computing system (e.g., backend computing system 108 in FIG. 1) (block 906). The backend computing then obtains an ambient condition value (block 908), for example, from the external computing device or from an external monitor, as discussed above. Alternatively, the backend computing system can receive the ambient condition value from another source, such as another computing device in communication with the backend computing system, the Internet, or some other source. The means by which the backend computing system obtains the ambient condition value is not critical, and thus, the present invention is not limited to any particular method or system for obtaining the ambient condition value. Also, as one skilled in the art will appreciate, backend computing system can obtain the ambient condition value prior to receiving the absolute physiologic parameter value. In addition, as discussed above, the physiologic parameter value and/or the ambient condition value can include time stamps, so that the relative physiologic parameter value is calculated using values that are similar in time.

After the backend computing system obtains the ambient condition value and the absolute physiologic parameter value, the backend computing system calculates a relative physiologic parameter value (block 910), and then the backend computing system stores the relative physiologic parameter value, for example, in a database or other similar storage area (block 912). The data then can be made available for later processing, access, and/or analysis by one or more health care providers, such as physicians, specialists, IMD manufacturers, insurance companies, pharmaceutical companies, or any other suitable health care provider.

After the relative physiologic parameter value is calculated at the backend computing system, additional processing can occur in a number of different devices in the system. This is illustrated by continuation block 914 in FIG. 9.

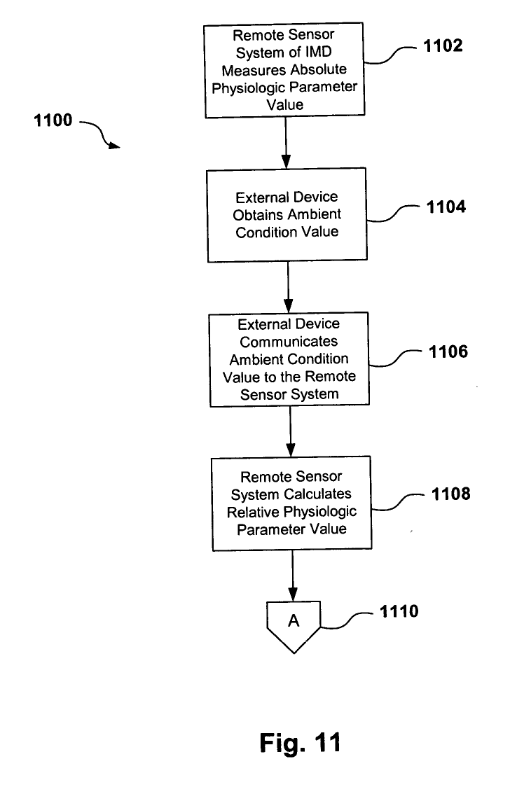

Referring now to FIGS. 10a-10d, additional embodiments of the present invention will be described with reference to flow charts 1000, 1010, 1020 and 1030, respectively. In FIGS. 10a-10b, continuation blocks 1002 and 1032 are continuations from block 914 in FIG. 9. Thus, in accordance with the embodiment illustrated in FIG. 10a, after the backend computing system calculates the relative physiologic parameter value, it communicates the relative physiologic parameter value to the external computing device. As discussed above, the external computing device then can display or provide the relative physiologic measurement values to a health care provider or other entity for review.

In addition, as illustrated by flow chart 1000 in FIG. 10a, the external computing device also can communicate the relative physiologic parameter value to the IMD (block 1006), which then can store the relative physiologic parameter value, for example, in a memory. When the IMD receives the relative physiologic parameter value, the IMD then can use the value for additional processing, as illustrated by continuation block 1008. Continuation blocks 1012 and 1022 in FIGS. 10b and 10c, respectively, continue from block 1008.

Referring now to FIG. 10b, in this particular embodiment, the IMD can be configured to calculate or determine a therapy based on the relative physiologic parameter value (block 1014). As discussed above, the IMD can be configured to administer the therapy to the patient (block 1016).

In an alternative embodiment illustrated in FIG. 10c, the IMD again can be configured to calculate or determine a therapy or therapy information based on the relative physiologic parameter value (block 1024), but in stead of, or in addition to administering the therapy to the patient, the IMD is configured to communicate the therapy information to the external computing device or external monitor (block 1026), which can be configured to provide or communicate the therapy information to the patient (block 1028).

Further, as discussed above, after the external computing device (and/or the external monitor) receives the therapy information from the IMD, a physician or other health care provider then can analyze the therapy information and provide it to the patient if the physician determines that it is appropriate, or the physician could provide different or additional therapy to the patient if the physician determines that an alternative therapy would be better. The therapy information also can be uploaded to a backend computing system (e.g., backend computing system 108) (block 1029) for later processing, access and/or analysis. In addition, in an alternative embodiment, instead of the IMD determining the therapy information, the external computing device and/or the external monitor can be configured to calculate or determine the therapy information.

Referring now to FIG. 10d, flowchart 1030 illustrates another embodiment of the present invention. In accordance with this particular embodiment, after the backend computing system calculates the relative physiologic parameter value, the backend computing system then can use that value to determine a therapy or therapy information for the patient (block 1034). The backend system then can store the therapy information in the database for later access by a health care provider (block 1036). In addition, the backend system can communicate the therapy information to the external computing device and/or the external monitor, which can display or provide the therapy information to a health care provider, as discussed above.

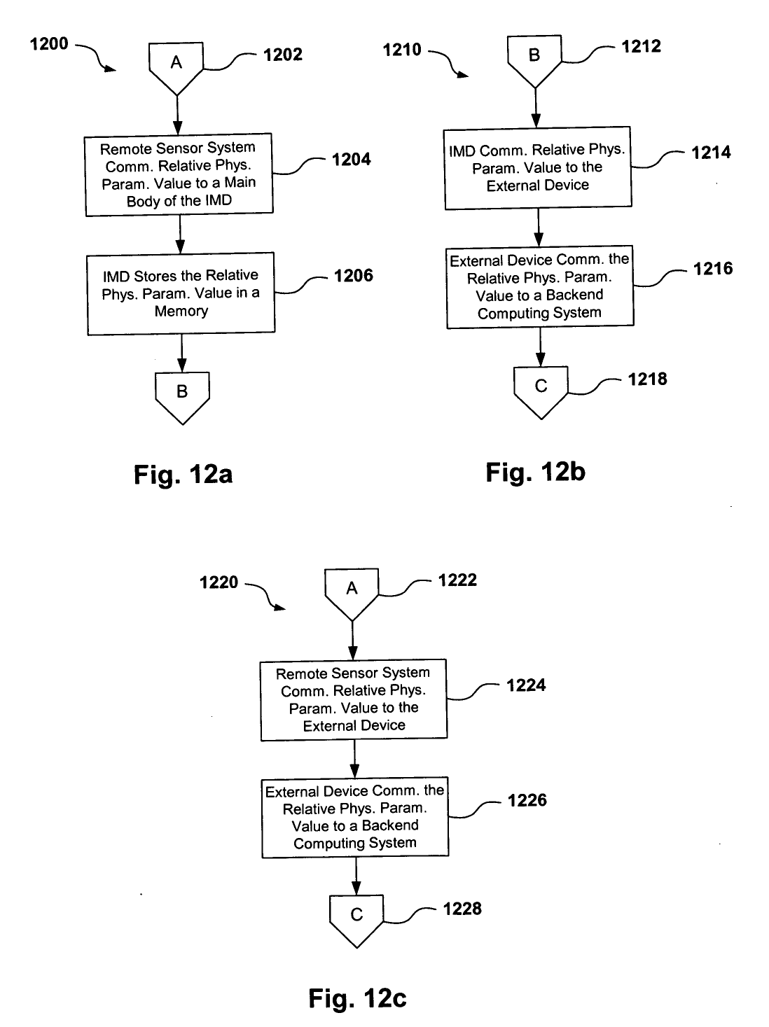

Referring now to FIG. 11, still another embodiment of a method of deriving and using relative physiologic parameter values is shown. In accordance with the method illustrated in flow chart 1100, a remote sensor system of an IMD (e.g., remote sensor system 112 in FIG. 1) measures an absolute physiologic parameter value (block 1102). As discussed above, the remote sensor system can comprises satellite sensors, which can measure any physiologic measurement, such as, blood pressure, temperature, blood or fluid flow, strain, electrical, chemical or magnetic properties within the body, or the like. In one particular embodiment, the physiologic parameter value obtained is blood pressure (e.g., intravascular or intracardiac blood pressure), and one or more pressure sensors associated with the remoter sensor system are used to obtain the absolute blood pressure value. Again, the absolute pressure measured by a pressure sensor can be adjusted for internal temperatures and/or a built-in backing pressure and/or temperature coefficients of the pressure sensor.

After the remote sensor system of the IMD obtains the absolute physiologic parameter (e.g., absolute blood pressure), an external device obtains an ambient condition value (block 1104). In one embodiment, the external device is an external monitor (e.g., external monitor 104 in FIG. 1), which includes one or more sensors for measuring the ambient condition value. In another embodiment, the external device is an external computing device (e.g., external computing device 106 in FIG. 1), which can either measure the ambient condition value, or obtain the ambient condition value from another source, such as a backend computing system, other sensors in communication with the external computing device, or the Internet.

After the external device obtains the ambient condition value, it communicates the ambient condition value to the remote sensor system (block 1 106), which then calculates a relative physiologic parameter value from the absolute physiologic parameter value and the ambient condition value (block 1108). As with other communication connections, the external device and the remote sensor system can implement a data transmission check (e.g., a cyclic redundancy code (“CRC”) check) to ensure the data is transmitted properly. Further, as discussed above, the physiologic parameter value and/or the ambient condition value can include time stamps, so that the relative physiologic parameter value is calculated using values that are similar in time. After the remote sensor system calculates the relative physiologic parameter value, additional processing can occur in a number of different devices in the system. This is illustrated by continuation block 1110 in FIG. 11.

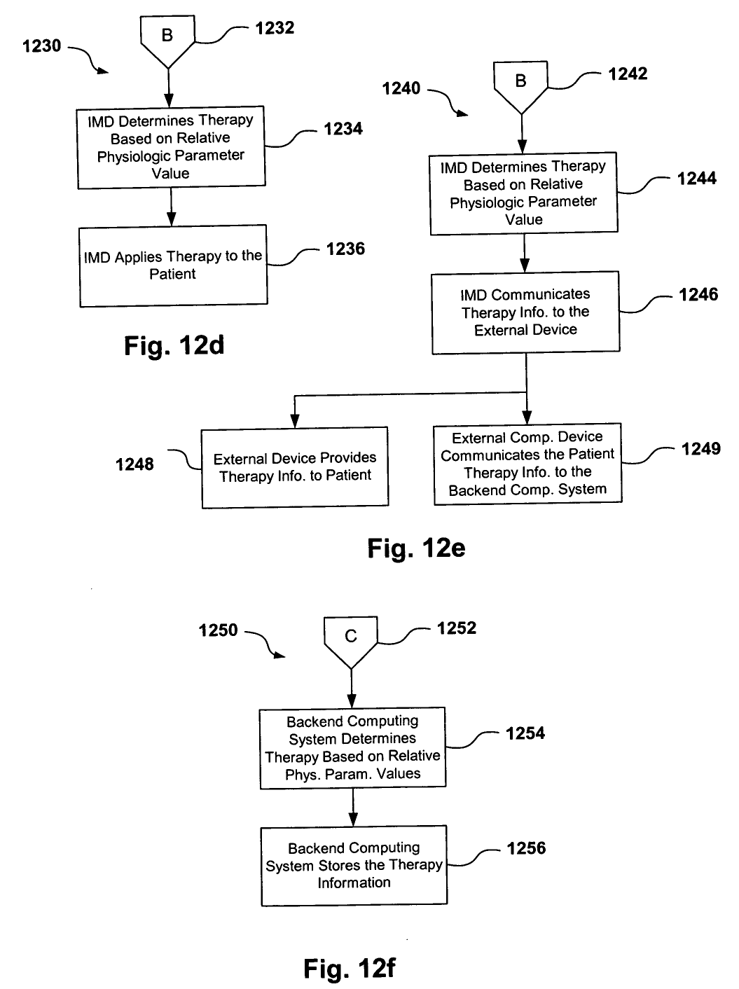

Referring now to FIGS. 12a-12f, additional embodiments of the present invention will be described with reference to flow charts 1200-1250. In accordance with the embodiment illustrated in FIG. 12a, after the remote sensor system calculates the relative physiologic parameter value, it communicates the relative physiologic parameter value to the main body of the IMD (e.g., IMD 102) (block 1204), which then can store the relative physiologic parameter value, for example, in a memory (block 1206).

In addition, after the IMD receives the relative physiologic parameter value, it can communicate it to the external device (block 1214 in FIG. 12b). As discussed in previous embodiments, the external device can provide the relative physiologic parameter value to the patient or a health care provider, or it can communicate the relative physiologic parameter value to a backend computing system (block 1216).

In another embodiment, instead of the main body of the IMD communicating the relative physiologic parameter value to the external device, the remote sensor system can be configured to communicate the relative physiologic parameter value to the external device directly (block 1224 in FIG. 12c). Then, as with the embodiment of FIG. 12b, the external device can display the relative physiologic parameter value, and/or it can communicate the value to a backend computing system (block 1226).

Upon receiving the relative physiologic parameter value, the backend computing system then can store the value in a database for access by health care providers. In addition, as discussed above, the backend computing system can use the relative physiologic parameter value to determine a therapy for the patient (block 1254 in FIG. 12f). The backend computing system then can store the therapy information in a database (block 1256), and/or the backend system can communicate the therapy information to the external device, which can display or provide the therapy information to relevant parties.

Referring now to FIGS. 12d and 12e, in accordance with other embodiments of the invention, after the IMD stores the relative physiologic parameter value (block 1206 in FIG. 12a), the IMD can be configured to calculate or determine a therapy based on the relative physiologic parameter value (blocks 1234 and 1244 in FIGS. 12d and 12e, respectively). As discussed above, the IMD can be configured to administer the therapy to the patient (block 1236), and/or the IMD can communicate the therapy information to the external device (block 1246). The external device then can provide or communicate the therapy information to the patient, a physician or other health care provider (block 1248). In addition, the therapy information can be uploaded to a backend computing system (block 1249) for later processing, access and/or analysis. In still other embodiments, instead of the backend computing system or the IMD determining the therapy information, the external device can be configured to calculate or determine the therapy.

In conclusion, the present invention provides novel systems, methods and arrangements for deriving relative physiologic parameter values from measured physiologic parameters and from ambient condition values. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A system for deriving relative physiologic measurement values, comprising:
an implantable medical device (“IMD”) operable to determine an absolute physiologic parameter value within a patient's body, and communicate the absolute physiologic parameter value outside the patient's body; and
an external monitor operable to:
receive the absolute physiologic parameter from the IMD;
obtain an ambient condition value outside the body that can affect the absolute physiologic parameter value; and
calculate a relative physiologic parameter value from the ambient condition value and the absolute physiologic parameter value;
wherein the external monitor is further operable to communicate the relative physiologic parameter value to the IMD, and wherein the IMD is further operable to receive the relative physiologic parameter value and store it in a memory.

2. The system as recited in claim 1, wherein the absolute physiologic parameter value is marked with a time stamp, and wherein the external monitor selects an ambient condition value that is obtained at a time reasonably close to the time stamp time to calculate the relative physiologic parameter value.

3. The system as recited in claim 1, wherein the IMD is further operable to communicate the relative physiologic parameter value to an external computing device.

4. The system as recited in claim 1, wherein the external monitor is further operable to store the relative physiologic parameter value in a memory.

5. The system as recited in claim 1, wherein the absolute physiologic parameter value comprises a blood pressure value, wherein the ambient condition value comprises an ambient pressure value, and wherein the relative physiologic parameter value comprises the blood pressure value relative to the ambient pressure value.

6. The system as recited in claim 5, wherein the relative physiologic parameter value is calculated by subtracting the ambient pressure value from the blood pressure value.

7. The system as recited in claim 5, wherein the blood pressure value is determined by:
measuring the blood pressure value with a pressure sensor; and
adjusting the measured blood pressure value with a sensor specific adjustment factor to obtain an absolute blood pressure value.

8. The system as recited in claim 5, wherein the blood pressure can be intravascular or intracardiac blood pressure.

9. The system as recited in claim 1, wherein the external monitor is operable to measure the ambient condition value.

10. The system as recited in claim 1, wherein the external monitor is operable to receive the ambient condition value from an ambient condition source.

11. The system as recited in claim 1, wherein the external monitor comprises a wireless communication device carried on or near the patient's body.

12. The system as recited in claim 11, wherein the external monitor is a monitor device selected from the group consisting of: a monitor device adapted to be worn around a patient's body part; a monitor device that can be connected to a belt; a monitor device that can be worn as a patch; a personal digital assistant (PDA); a monitor device that can be carried in a pocket or a personal carry bag; and a customized cell phone or pager.

13. The system as recited in claim 1, wherein the external monitor comprises an external computing device selected from the group consisting of a personal computer, an IMD programmer, an IMD repeater device located in a patients residence, and a backend computing system.

14. The system as recited in claim 1, wherein the IMD and the external monitor are operable to communicate via a wireless communication connection selected from the group consisting of: a radio frequency communication connection; an acoustic communication connection; an electric field communication connection; and an optical communication connection.

15. The system as recited in claim 1, wherein the IMD is selected from the group consisting of a physiologic parameter sensor, a pacemaker, a defibrillator, a bi-ventricular pacer, a ventricular assist blood pump, a drug delivery pump, a drug infusion device, a neurostimulating device, an intra-ocular shunt, and an intra-cranial shunt.

16. The system as recited in claim 1, wherein the IMD is operable to provide therapy to a patient, and wherein the IMD uses the relative physiologic parameter value to determine the proper therapy to provide to the patient.

17. The system as recited in claim 16, wherein the therapy is selected from the group consisting of cardiac pacing therapy, anti-tachycardia therapy, drug delivery therapy, neurostimulation therapy, and blood pump therapy.

18. The system as recited in claim 1, wherein:
the IMD comprises a processor operable to generate patient therapy information based at least in part on the relative physiologic parameter value;
the IMD is further operable to communicate the patient therapy information to the external monitor; and
the external monitor comprises a patient communication interface operable to provide the patient therapy information to the patient.

19. A method for deriving relative physiologic measurement values, comprising:
an implantable medical device (“IMD”) determining an absolute physiologic parameter value within a patient's body;
communicating the absolute physiologic parameter value to an external monitor outside the patient's body;
the external monitor obtaining an ambient condition value outside the body that can affect the absolute physiologic parameter value;
calculating a relative physiologic parameter value from the ambient condition value and the absolute physiologic parameter value;
communicating the relative physiologic parameter value from the external monitor to the IMD, and
storing the relative physiologic parameter value in a memory in the IMD.

20. The method as recited in claim 19, further comprising the steps of:
the IMD marking the absolute physiologic parameter value with a time stamp; and
the external monitor selecting an ambient condition value that is obtained at a time reasonably close to the time stamp time to calculate the relative physiologic parameter value.

21. The method as recited in claim 19, further comprising the step of-communicating the relative physiologic parameter value from the IMD to an external computing device.

22. The method as recited in claim 19, further comprising the step of the external monitor storing the relative physiologic parameter value in a memory.

23. The method as recited in claim 19, wherein the absolute physiologic parameter value comprises a blood pressure value, wherein the ambient condition value comprises an ambient pressure value, and wherein the relative physiologic parameter value comprises the blood pressure value relative to the ambient pressure.

24. The method as recited in claim 23, wherein the step of calculating a relative physiologic parameter value comprises subtracting the ambient pressure value from the blood pressure value.

25. The method as recited in claim 23, wherein the step of determining the blood pressure value, comprises:
measuring the blood pressure value with a pressure sensor; and
adjusting the blood pressure value with a sensor specific adjustment factor to obtain an absolute blood pressure value.

26. The method as recited in claim 23, wherein the blood pressure can be intravascular or intracardiac blood pressure.

27. The method as recited in claim 19, wherein the step of the external monitor obtaining an ambient condition value comprises the external monitor measuring the ambient condition value.

28. The method as recited in claim 19, wherein the step of the external monitor obtaining an ambient condition value comprises the external monitor receiving the ambient condition value from an ambient condition source.

29. The method as recited in claim 19, wherein the external monitor comprises a wireless communication device carried on or near the patient's body.

30. The method as recited in claim 29, wherein the external monitor is a monitor device selected from the group consisting of: a monitor device adapted to be worn around a patient's body part; a monitor device that can be connected to a belt; a monitor device that can be worn as a patch; a personal digital assistant (PDA); a monitor device that can be carried in a pocket or a personal carry bag; and a customized cell phone or pager.

31. The method as recited in claim 19, wherein the external monitor comprises an external computing device selected from the group consisting of a personal computer, an IMD programmer, an IMD repeater device located in a patients residence, and a backend computing system.

32. The method as recited in claim 19, wherein the steps of communicating the measured absolute physiologic parameter value to an external monitor and communicating the relative physiologic parameter value from the external monitor to the IMD comprise communicating via a wireless communication connection selected from the group consisting of: a radio frequency communication connection; an acoustic communication connection; an electric field communication connection; and an optical communication connection.

33. The method as recited in claim 19, wherein the IMD is selected from the group consisting of a physiologic parameter sensor, a pacemaker, a defibrillator, a bi-ventricular pacer, a ventricular assist blood pump, a drug delivery pump, a drug infusion device, a neurostimulating device, an intra-ocular shunt, and an intra-cranial shunt.

34. The method as recited in claim 19, further comprising:
the IMD determining a proper therapy to provide to the patient using the relative physiologic parameter value; and
the IMD providing the proper therapy to the patient.

35. The method as recited in claim 34, wherein the therapy is selected from the group consisting of cardiac pacing therapy, anti-tachycardia therapy, drug delivery therapy, neurostimulation therapy, and blood pump therapy.

36. The method as recited in claim 19, further comprising:
the IMD generating patient therapy information based at least in part on the relative physiologic parameter value;
communicating the patient therapy information from the IMD to the external monitor; and
the external monitor providing the patient therapy information to the patient via a communication interface.

37. A system for deriving relative blood pressure values, comprising:
an implantable medical device (“IMD”) operable to determine blood pressure within a patient's body, and communicate the blood pressure outside the patient's body; and
an external monitor operable to:
receive the blood pressure from the IMD;
obtain an ambient pressure value outside the body; and
calculate a relative blood pressure value from the ambient pressure value and the blood pressure;
wherein the external monitor is further operable to communicate the relative blood pressure value to the IMD, and wherein the IMD is further operable to receive the relative blood pressure and store it in a memory.

38. The system as recited in claim 37, wherein the blood pressure can be intravascular or intracardiac blood pressure.

39. The system as recited in claim 37, wherein the absolute physiologic parameter value is marked with a time stamp, and wherein the external monitor selects an ambient condition value that is obtained at a time reasonably close to the time stamp time to calculate the relative physiologic parameter value.

40. The system as recited in claim 37, wherein the IMD is operable to provide therapy to a patient, and wherein the IMD uses the relative blood pressure value to determine the proper therapy to provide to the patient.

41. The system as recited in claim 37, wherein:
the IMD comprises a processor operable to generate patient therapy information based at least in part on the relative blood pressure value; and
the IMD is further operable to communicate the patient therapy information to an external communication device operable to provide the therapy information to the patient or a health care provider.
